(19) United States
(12) Patent Application Publication
(10) Pub. No.: US 2022/0104797 A1
(43) Pub. Date: Apr. 7, 2022

(54) LUNG BIOPSY DEVICES, SYSTEMS AND METHODS FOR LOCATING AND BIOPSYING AN OBJECT

(71) Applicant: Regents of the University of Minnesota, Minneapolis, MN (US)

(72) Inventors: Michael A. Greminger, Duluth, MN (US); Anastasia Zink, Minneapolis, MN (US); Brian Krohn, Saint Paul, MN (US); Amit Goyal, St. Anthony, MN (US); Roy Joseph Cho, Edina, MN (US); H. Erhan Dincer, Saint Paul, MN (US); Felix Zamora, Albuquerque, NM (US); Gills Fai, Duluth, MN (US); Sarah Ostlie, Roseville, MN (US)

(21) Appl. No.: 17/310,533

(22) PCT Filed: Feb. 7, 2020
(86) PCT No.: PCT/US20/17132

§ 371 Date: Aug. 9, 2021

Related U.S. Application Data
(60) Provisional application No. 62/802,787, filed on Feb. 8, 2019.

(51) Int. Cl. A61B 10/04; A61B 17/34

(52) U.S. Cl. CPC A61B 10/04; A61B 2017/00026; A61B 2010/045; A61B 17/3403

(57) ABSTRACT

The disclosure describes a lung biopsy system including an elongated housing extending from a proximal end to a distal end, at least one biopsy needle guide lumen extending within the housing, the guide lumen configured to enable deployment of a biopsy needle and extending from a proximal portion of the housing to a biopsy port in the housing, the biopsy port being positioned proximal to the distal end, a camera configured to be arranged in the housing to obtain optical data relating to a position of the system, a light source configured to be arranged in the housing adjacent to the camera to produce illumination for the camera; and one or more electrodes carried by the housing and positioned adjacent to the biopsy port, the one or more electrodes configured to measure an impedance of a surrounding tissue of a patient.

### RELATED APPLICATION

This application claims the benefit of U.S. Provisional Application No. 62/802,787 filed on Feb. 8, 2019, the entirety of which is incorporated herein by reference.

### TECHNICAL FIELD

Examples relate generally to medical examinations and biopsies of the body and more particularly to devices, systems and methods for lung and other biopsies including visual or photographical inspection.

### BACKGROUND

Lung cancer is the leading cause of cancer deaths worldwide. There were 180,000 Medicare/Medicaid admissions for bronchial or lung cancer in 2010. In 2013, Centers for Medicare & Medicaid Services paid for 46,000 bronchoscopic lung biopsy procedures. Of these procedures, 3,400 procedures were performed using electromagnetic navigation bronchoscopy (ENB), and 15,300 procedures were performed using endobronchial ultrasound (EBUS).

ENB can be used to track tools within the body using radio waves. Antennas are placed on the patient's body, which receive a signal emitted from the tools inserted into the body. The position of the tool is superimposed onto a CT image of the patient to provide feedback to the physician on the location of the tool. There are at least two ENB systems currently on the market. One such system, the SuperDimension system, is cost-prohibitive for many clinics, at several hundred thousand dollars per unit. A competitor to SuperDimension is the Veran Medical EndoBronchial guidance system, which gained FDA clearance in 2009. The SuperDimension system has a steerable guide catheter with position sensor that is smaller than a bronchoscope. Once the guide catheter is placed, a tool can be deployed through the guide catheter. The Veran Medical system adds some additional features, and supplies a suite of tools that all include the position-sensing element at the tip of the tool. They enable the switching from an endobronchial approach to a transcutaneous approach in a single setup. The Veran Medical system likewise can also be cost-prohibited to many clinics or hospitals.

If a transbronchial approach cannot be used to obtain a biopsy, a transthoracic needle can be used to acquire a sample. In 2013, 40,000 transthoracic needle biopsies were paid for by Centers for Medicare & Medicaid Services. The risk of pnuemothorax for transthoracic needle biopsies is between 9 and 54%, however.

EBUS avoids the necessity for use of a transthoracic needle in some instances, where the object to be biopsied is located on a main bronchial lumen having a relatively large diameter. EBUS involves using an ultrasonic sensor on a bronchoscope to determine when the bronchoscope is passing by an object having different density or acoustical properties from the rest of the lung. For example, a tumor or nodule can be detected by the difference in acoustical response. The bronchoscope can also include a biopsy needle, such that the detected tumor, nodule, or other object can be biopsied.

EBUS can be implemented on bronchoscopes that are used to biopsy tumors that are in the larger bronchial lumens of the lung. A typical bronchoscope, having a 5.3 millimeter (mm) standard outer diameter, cannot reach the periphery of the lung, where the bronchial lumens are less than 5.3 mm wide. If the object is adjacent to a large bronchial lumen, the EBUS can be routed to it. The edges of the tumor can be seen in the EBUS image generated from ultrasound data.

Existing ENB systems are expensive and often cost-prohibitive for smaller hospitals or clinics where they would not be used routinely. Furthermore, use of transthoracic needles can result in severe complications such as a collapsed lung. EBUS, while less expensive than ENB, is only suitable when the object to be biopsied is located on a main bronchial path having a large diameter that affords access to the bronchoscope, and much of the lung may be inaccessible to EBUS systems.

### SUMMARY

Devices, systems, and methods for locating and biopsying an object in a pulmonary system are disclosed. In examples, such a system does not require simultaneous electromagnetic navigation bronchoscopy (ENB) guidance or transthoracic puncture, and is capable of reaching objects adjacent to pulmonary passages that are smaller than those reachable by a standard bronchoscope. Systems discussed herein may include a tool that is small enough to be deployed by a bronchoscope tool port, and include a camera, light-emitting diode (LED), and sensor configured to detect abnormal tissue.

In one example, a system includes a housing, a camera, a light source, and one or more electrodes on the housing. The housing extends from a proximal end to a distal end. The camera is arranged within the housing at the distal end to obtain optical data relating to the position of the system. The light source is arranged within the housing opposite the camera from the distal end. The light source is then configured to produce illumination passing between the housing and the camera at the distal end. The one or more electrodes are configured to measure the impedance within the surrounding tissue (e.g., lung tissue) to distinguish between normal and abnormal lung tissue. A biopsy needle guide lumen within the hosing such that the distal end of the guide lumen is positioned adjacent to the one or more electrodes thereby forming a port (e.g., biopsy port) within the housing. The guide lumen is configured to allow a biopsy needle to pass through the lumen and be deployed from the lumen through the biopsy port. The biopsy needle can be bendable, such that the biopsy needle deployed from the biopsy needle guide lumen through biopsy port is able to take a biopsy at the position where the one or more electrodes detected abnormal tissue.

In some examples, the disclosure describes a lung biopsy system including an elongated housing extending from a proximal end to a distal end, at least one biopsy needle guide lumen extending within the housing, the guide lumen configured to enable deployment of a biopsy needle and extending from a proximal portion of the housing to a biopsy port in the housing, the biopsy port being positioned proximal to the distal end, a camera configured to be arranged in the housing to obtain optical data relating to a position of the system, a light source configured to be arranged in the housing adjacent to the camera to produce illumination for the camera; and one or more electrodes carried by the housing and positioned adjacent to the biopsy port, the one or more electrodes configured to measure an impedance of a surrounding tissue of a patient.

In some examples the disclosure describes a method that includes navigating a lung biopsy system through a pulmonary system of a patient, the lung biopsy system including an elongated housing extending from a proximal end to a distal end, at least one biopsy needle guide lumen extending within the housing, the guide lumen configured to enable deployment of a biopsy needle and extending from a proximal portion of the housing to a biopsy port in the housing, the biopsy port being positioned proximal to the distal end, a camera configured to be arranged in the housing to obtain optical data relating to a position of the system, a light source configured to be arranged in the housing adjacent to the camera to produce illumination for the camera, and one or more electrodes carried by the housing and positioned adjacent to the biopsy port, the one or more electrodes configured to measure an impedance of a surrounding tissue of a patient adjacent to the one or more electrodes. The method further including using the one or more electrodes, measuring the impedance within the surrounding tissue to detect relative changes in the impedance, and deploying a biopsy needle through the biopsy needle guide lumen and out the biopsy port to obtain a tissue sample from the surrounding tissue that is adjacent to the one or more electrodes.

The above summary is not intended to describe each illustrated example or every implementation of the subject matter hereof. The figures and the detailed description that follow more particularly exemplify various examples.

### BRIEF DESCRIPTION OF THE DRAWINGS

Examples may be more completely understood in consideration of the following detailed description of various examples of the invention in connection with the accompanying figures, in which:

FIGS. 1A and 1B are perspective views of an example dual-needle lung biopsy tool according to the disclosure.

FIG. 1C is a side view of the lung biopsy tool of FIGS. 1A and 1B, from the distal end.

FIG. 1D is a flowchart of an example method for operating the system of FIGS. 1A-1C.

FIGS. 2A and 2B are perspective views of an example single-needle lung biopsy tool according to the disclosure.

FIGS. 3A and 3B are schematic views of an example single-needle lung biopsy tool with a separate lumen for an ultrasonic sensor according to the disclosure.

FIGS. 4A-4C are schematic views of an example single-needle lung biopsy tool with a separate lumen that allows an ultrasonic sensor to be interchanged with a camera and LED module, according to the disclosure.

FIG. 5 is a perspective view of an example deformable lung biopsy tool positioned in a bronchoscope according to the disclosure.

FIGS. 6A-6D are schematic views depicting an example system for deforming a lung biopsy tool according to the disclosure.

FIG. 7 is a schematic view depicting an example coil spring system for preventing kinking and facilitating straightening of a lung biopsy tool according to the disclosure.

FIGS. 8A and 8B are perspective and side views of an example single-needle lung biopsy tool according to the disclosure.

FIG. 8C is a perspective view of a single-needle lung biopsy tool according to another embodiment.

While various examples are amenable to various modifications and alternative forms, specifics thereof have been shown by way of example in the drawings and will be described in detail. It should be understood, however, that the intention is not to limit the claimed inventions to the particular examples described. On the contrary, the intention is to cover all modifications, equivalents, and alternatives falling within the spirit and scope of the subject matter as defined by the claims.

### DETAILED DESCRIPTION OF THE DRAWINGS

Examples discussed herein include a lung biopsy system that can be deployed from a standard tool port of a bronchoscope (e.g., having a diameter of about 2 mm or less) or can be deployed independently. Within this system are arranged, in order from the distal end of the system in one example: a camera, an LED, and a sensor configured to detect abnormal lung tissue. In other examples, the sensor may be positioned adjacent to, or may be interchangeable with, the camera and LED. The system can also include a biopsy needle guide lumen and port that facilitates deployment of a biopsy needle adjacent to the sensor once abnormal tissue is detected. Such components can be used to optically guide the system to an object in a pulmonary system to acquire a biopsy sample, even if that object is not located in a large, primary pulmonary passageway that is reachable by the relatively larger bronchoscope.

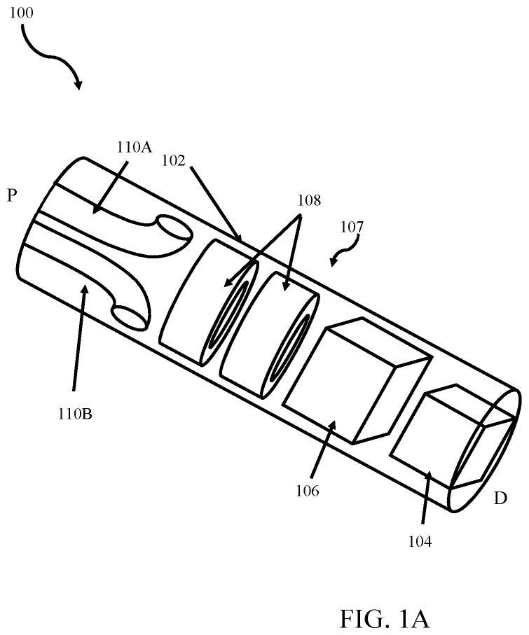

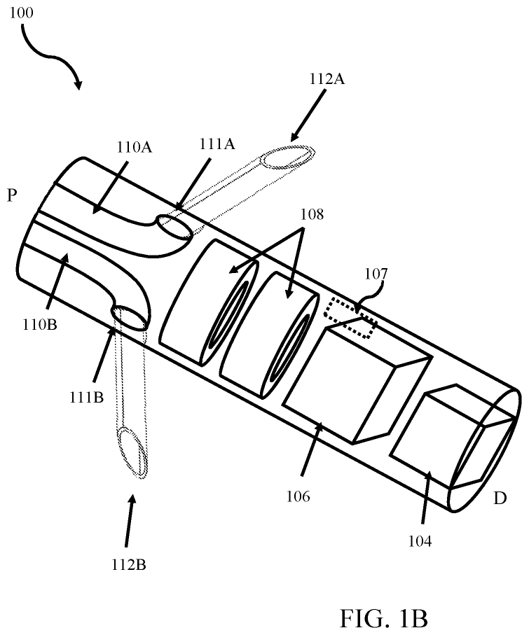

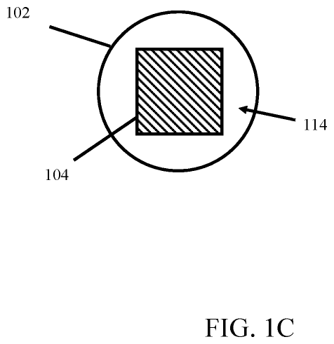

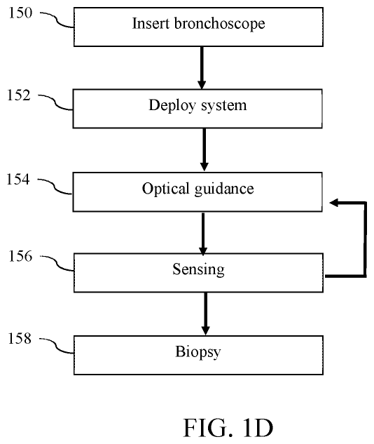

FIGS. 1A and 1B are perspective views of an example lung biopsy system 100 according to the disclosure. System 100 includes a housing 102, a camera 104, a light-emitting diode (LED) 106, a sensor 107, a first biopsy needle guide lumen 110A having a first needle biopsy port 111A, and a second biopsy needle guide lumen 110B having a second needle biopsy port 111B. FIG. 1B additionally depicts a first biopsy needle 112A and a second biopsy needle 112B.

System 100 is sized for deployment via a bronchoscope to a remote location of a pulmonary system. Bronchoscopes are standardized devices with an outer diameter of about 5.3 mm. Within each bronchoscope are several standard ports, including a tool port having a diameter of about 2 mm. A bronchoscope can be used to enter a pulmonary system but often cannot be extended all the way to an object that a physician wishes to biopsy because the pulmonary passageways are not wide enough for the bronchoscope to pass through. Rather than access the object using a transthoracic needle, system 100 can be deployed from the tool port of a bronchoscope and, due to its relatively small outer diameter, routed more easily to objects that are positioned along pulmonary passageways having relatively small diameters.

System 100 includes an elongated body that extends along a longitudinal axis from a proximal end to a distal end D. The proximal side of the devices illustrated in the accompanying figures is indicated by the notation P. The devices are not drawn to scale. The proximal side P and proximal end of system 100 may include any suitable arrangement of features that can be allow system 100 to be manipulated by the physician at the bronchoscope and/or outside (external) of the patient. Distal end D of system 100 can be driven through pulmonary passageways to various target sites.

The elongated body of system 100 includes housing 102 that forms an outer wall of system 100, housing the other components of system 100. Housing 102 can be made from any suitable material including for example, a flexible material such as a polymer. In some examples, it may be desirable for housing 102 to be at least partially made of a transparent or translucent material, such that light can pass through housing 102, particularly at portions of housing adjacent or distal to camera 104 and LED 106. Housing 102 extends from a proximal end (at or near the bronchoscope; not shown) to a distal end D (at or near camera 104). In some examples, housing 102 may include support structures (e.g., coils, braids, or wires) or may be comprised of one of more layers that may help with the navigation of or strength of system 100.

System 100 also includes camera 104 arranged at or near the distal end D of housing 102. Camera 104 can be used to provide an indication of the position of system 100 in a pulmonary system. Camera 104 can send back signals to the physician that show what is in front of system 100, either wirelessly or through a wired connection (not shown) extending from the proximal end to the distal end D of system 100. Camera 104 can provide feedback on the actual position of system 100 in the pulmonary passage. This may solve one or more problems of conventional systems, in that navigational feedback from EBUS or ENB systems can be incorrect and make advancement difficult.

In some examples, camera 104 can be a 1 mm×1 mm square camera. In other examples, the size and shape of camera 104 can vary. For example, in alternative examples camera 104 could have a round profile from distal end D. Camera 104 is small enough, however, that when positioned in housing 102 having a circular outer diameter, at least some portion of the distal end D of housing 102 is not overlapped by camera 104, as shown in FIG. 1C.

System 100 may also includes LED 106 positioned in close relative proximity to camera 104. In some examples, LED 106 may be positioned proximal to camera 104 and define a larger cross-sectional area relative to the longitudinal axis of system 100 than camera 104 when viewed from the distal end D. In this way, light from LED 106 projected distally passes around camera 104 and out the distal end D of system 100, lighting the pulmonary passageways so that camera 104 can be used to provide information about the position of system 100. In examples where the material making up housing 102 is transparent or translucent, light from LED 106 or another light source can pass through the distal end D of system 100. In other examples, LED 106 may not have a larger cross-sectional area than camera 104. For example, outer housing 102 can be made of a transparent or translucent matter, as described above. In those examples, light can be routed, through either reflections or refractions, around camera 104. Light can therefore be directed toward the distal end D of system 100 to illuminate the pulmonary passageways for camera 104. In alternative examples, LED 106 could be replaced by some other source of illumination. In examples, the illumination provided by LED 106 or another light source could be at an angle and wavelength that promotes total internal reflection within the material that makes up outer housing 102 or another transmissive material arranged between camera 104 and outer housing 102. In examples, LED 106 or another source of illumination is tuned to the wavelengths or color spectra corresponding to those that are easily detected by camera 104. Additionally or alternatively, LED 106 or another source of illumination can be tuned to a wavelength that is primarily reflected from pulmonary passages rather than absorbed.

In some examples, housing 102 can be one or more layers of a molded or extruded polymer that encapsulates or surrounds both camera 104 and LED 106, and outer housing 102 can act as a carrier for the light produced by LED 106. In other examples, alternative systems for delivering light to the distal end D of system 100 could be used. For example, fiber optics or other light guides could be used to route light from LED 106 or other illumination devices toward the distal end D of system 100.

System 100 also includes a sensor 107 configured to detect abnormal tissue within the pulmonary passageways. In some examples, sensor 107 may include an ultrasonic sensor arranged along system 100 coaxially with camera 104 and LED 106. This coaxial arrangement allows outer housing 102 to be substantially narrower than if the same components were arranged in another fashion (e.g., within the same cross-sectional plane). The ultrasonic sensor can emit and/or detect ultrasonic signal passing through adjacent objects, such as the pulmonary passageway. Tumors, nodules, or other objects and abnormalities that a physician may wish to biopsy often have different acoustical properties in the ultrasonic frequencies than the rest of a pulmonary passageway. Using the signal detected by the ultrasonic sensor, a physician can determine that the ultrasonic sensor is adjacent to the object of interest for biopsy.

Ultrasonic sensors, while comparatively less expensive than ENB systems, may still be one of the more costly components in system 100, which may be less desirable, particularly if system 100 is configured to be disposable or a one-time use item. In other examples, sensor 107 may include an alternative sensing device. For example, as shown in FIG. 1A, sensor 107 may include one or more electrodes 108. The construction and materials used to form electrodes 108 may be less expensive than those used to form and include ultrasonic sensors. The inclusion of electrodes 108 and exclusion of an ultrasonic sensor from system 100 may therefore help reduce the cost and/or improve the disposability of the device. Additionally, or alternatively, the components used to form electrodes 108 may be less complex than those used to form an ultrasonic sensor and may take up less space within system 100, thereby allowing the diameter of housing 102 to be comparatively smaller. Further, electrodes 108 have the added potential of being able to take into account impedance related to air gaps within the tissue or body.

Electrodes 108 may be configured to measure the relative impedance of the surrounding lung tissue. For example, by passing oscillating voltage across two or more pairs of electrodes (e.g., a primary electrode and a secondary electrode) the phase and amplitude of the current that flows between the pair of electrodes can be used to determine the impedance of the surrounding tissue. This impedance has resistive, capacitive, and inductive components. The resultant impedance of the surrounding tissue will depend of the conductivity and permittivity of the tissue and/or other materials surrounding electrodes 108. The relative density, makeup, surrounding environment, and other physical properties of the surrounding tissue may all contribute to the resultant impedance measured. Accordingly, the measured impedance for lung nodules, tumors, and the like will differ compared to the impedance measured for otherwise normal and healthy lung tissue. For example, lung nodules or tumors are typically denser compared to healthy lung tissue. The impedance in such areas within the tissue containing such abnormalities therefore may be lower.

Additionally, system 100 may be used in areas of the lungs in which a bronchi in which system 100 is deployed is adjacent to another bronchi or tissue in which an abnormality may exist. In some embodiments, the measured impedance may make such an abnormality apparent, with the system accounting for air or other tissue between electrodes 108 and the abnormality. In this and other embodiments, ranges of impedances may be identified and associated with one or more suspected types of tissues, a likelihood that tissue is diseased vs. healthy, or some other characteristic(s). In some examples, cancerous lung tissue has a relative permittivity that is 1.2 to 3 times higher than healthy lung tissue. This will lead to a higher capacitance between electrodes 108 which will result in a lower imaginary part of the impedance. However, this change in impedance may not be proportional to the ratio of the permittivity values since it may also depend on the electrode geometry, the other tissues surrounding the tumor, and the parasitic capacitance of the electrode leads. In some examples, electrodes 108 may be covered or encapsulated with an insulating layer so that the impact of an air gap between the electrodes and the bronchiole wall will be reduced. Doing so may help ensure that the relative permittivity differences are being considered versus the conductivity differences between the materials. Additional details regarding relative permittivity differences between normal and cancerous tissue are described in the article Experimental Study of Dielectric Properties of Human Lung Tissue in Vitro, Wang et al., J. Med. Biol. Eng., Vol. 34 No. 6 (2014), pg. 598-604, which is incorporated by reference in its entirety.

In some examples, electrodes 108 may be interrogated to determine the impedance within different areas within the surrounding tissue to develop a two-dimensional mapping of the surrounding tissue. The interrogation may occur in real-time at the control of a physician, or may be automated by a computer system to measure the impedance at set or random time intervals. This impedance mapping may be used to detect areas of dense or abnormal tissue indicative of tumor growth. The automated computer system may use machine learning or suitable algorithms to analyze the impedance of the surrounding tissue and compare the impedance of different areas to each other and/or know standards to determine areas containing abnormal tissue (e.g., cancerous tumors). Such areas or regions of tissue may be flagged by the automated system to alert a clinician so that further biopsy of the area may be preformed. In some examples, the oscillating voltage may be a discrete or broadband frequency. Different frequency bands may be associated with different structural or functional aspects of target tissue as has been discussed by, for example, S. Kimura, T. Morimoto, T. Uyama, Y. Monden, Y. Kinouchi, and T. Iritani, “Application of Electrical Impedance Analysis for Diagnosis of a Pulmonary Mass,” CHEST, vol. 105, no. 6, pp. 1679-1682, June 1994.

In some examples, electrodes 108 may be used in conjunction with camera 104, an external ultrasound device (e.g., external to the patient), a computed tomography (CT) scan, or the like to detect the relative position of electrodes 108 within the pulmonary system of the patient. Such equipment may also be useful in assessing the position of system 100 and or the abnormal tissue region for the deployment of one or more of biopsy needles 112A and 112B into the targeted tissue area.

Electrodes 108 may each define a conductive surface configured to be brought into contact with the tissue of a patient. Suitable materials may include, for example, electrically conductive metals (e.g., stainless steel, nitinol, and the like), electrically conductive polymers, or the like. In some examples, electrodes 108 may include a multilayered construction using a combination of materials as know in the art. Additionally, or alternatively, electrodes 108 may be encapsulated in an electrically insulating material to make the device less sensitive to situations where an air gap may form between the electrodes and the bronchiole wall. With this approach, the relativity permittivity of the tissues is the main property that is being contrasted with the impedance measurement as opposed to the tissue electrical conductivity.

Electrodes 108 may be powered by one or more electrical conductors (not shown) that extend along the length of housing 102, which may be accessible at the proximal side of the device as shown by the position of electrode 107 of FIG. 1B. The electrical conductors may take on any suitable form. In some examples, the conductors may include one or more electrical wires having a distal end electrically coupled (e.g., welded or soldered) to one or more electrodes 108. The electrodes may be passed through an inner lumen of housing 102, embedded or secured to housing 102, or other suitable configuration. In some examples, the one or more electrical conductors may be in the form of a flexible circuit or ribbon wire attached to housing 102 (e.g., attached to the exterior of housing 102 or encased within a sheath). Additionally, or alternatively, the flexible circuit or ribbon wire may also be used to power one or more of LED 106 and camera 104.

Electrodes 108 may be incorporated into system 100 in any suitable number and arrangement. In some examples, system 100 may include at least one designated primary electrode and at least one designated secondary electrode. The characterization of an electrode as “primary” or “secondary” is used merely to distinguish the electrodes within a given pair of electrodes for purposes of completing the electrical pathway and is not intended to impose a structural preference or directionality of the current transmitted there between. In some examples, system 100 may include a plurality of respective primary electrodes and one or more secondary electrodes. In some such examples, a single secondary electrode may function as the corresponding paired electrode for some or all of the primary electrodes.

Electrodes 108 may take on any suitable shape or form. For example, electrodes 108 may be in the form of a ring electrode that is co-extruded, encapsulated, or secured to housing 102. In other examples, electrodes 108 may be paddle electrodes (e.g., a conductive surface on housing that does not completely encircle housing 102) or other suitable construction. A respective pair of electrodes 108 (e.g., the primary/transmitting electrode and the secondary/receiving) may be positioned along housing 102 at different relative positions along the longitudinal axis. In this manner, electrodes 108 may measure the impedance of tissue that lies between the relative positions of the pair of electrodes 108. In some examples, a respective pair of electrodes 108 may be separated by longitudinal distance of about 0.05 to 1 centimeters (cm) along housing 102.

In FIG. 1A, each of electrodes 108 are positioned distal to biopsy ports 111A and 111B and proximal to LED 106 and camera 104, however electrodes 108 may be positioned at any suitable point along housing 102. In general, it may be beneficial to have one or more pairs of electrodes 108 positioned in close proximity to both camera 104 and biopsy ports 111A and 111B. In this way, camera 104 may help to visualize some of the surrounding tissue to help discern some of the impedance measurements obtained by electrodes 108 and identify anomalous signals (e.g., signals indicating a branched region or air gap). Additionally, having electrodes 108 in close relative proximity to biopsy ports 111A and 111B may help to ensure that the biopsy sample being taken corresponds to the tissue region identified by electrodes 108 without needing to reposition system 100 relative to the surrounding tissue. In some examples, one or more of electrodes 108 may be positioned within a longitudinal distance of less than about 2 cm relative to camera 104, biopsy ports 111A and 111B, or both.

Electrodes 108 may be positioned distal to biopsy ports 111A and 111B, proximal to biopsy ports 111A and 111B, or a combination thereof. For example, for a respective pair of electrodes, one of the electrodes 108 may be positioned distal to biopsy ports 111A and 111B and one of the electrodes 108 may be poisoned proximal to biopsy ports 111A and 111B. In this configuration, the impedance measured by electrodes 108 may correspond to the tissue radially adjacent to biopsy ports 111A and 111B. In other examples, due to the curvature of biopsy guide lumens 110A and 110B and the angle at which biopsy needles 112A and 112B exit through biopsy ports 111A and 111B, it may be desirable to position electrodes 108 to measure the impedance of tissue more distal relative to biopsy ports 111A and 111B which will correspond to the tissue region that biopsy needles 112A and 112B will enter. As such, at least one or both of a respective pair of electrodes 108 may be positioned distal to biopsy ports 111A and 111B.

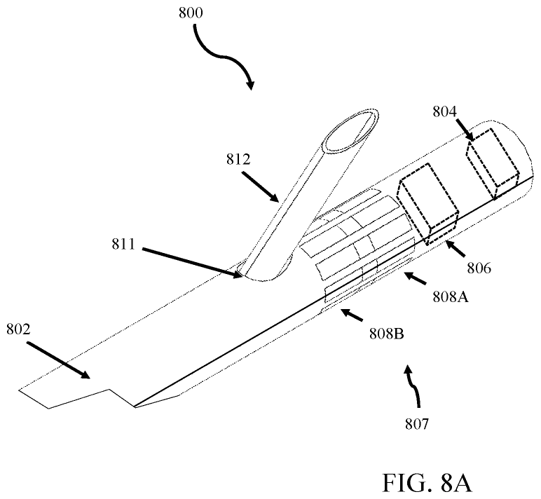

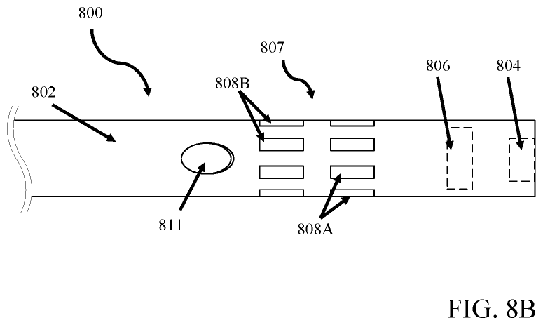

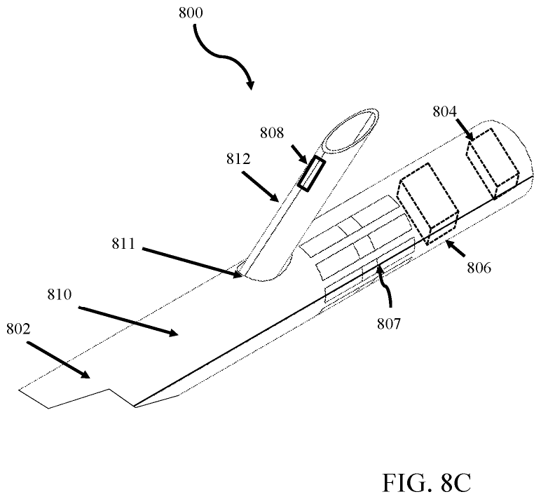

In some examples, as described further below with respect to FIGS. 8A-8B, system 100 may include a plurality of electrodes 108 (e.g., two or more). In some such examples, two or more of electrodes 108 may be positioned within the same cross-sectional plane relative to the longitudinal axis of housing 102. For example, independent primary electrodes may be positioned around the perimeter of housing 102 within the same general cross-sectional plane. Each primary electrode may be configured to communicate with a respective or universal secondary electrode to provide comparative impedance measurements of the surrounding tissue relative to the tissues radial position to housing 102. The radial component of the impedance measurements may help to orient housing 102 and biopsy ports 111A and 111B relative to a targeted tissue area.

System 100 also includes first biopsy needle guide lumen 110A and second biopsy needle guide lumen 110B extend within the region bounded by outer housing 102, from the proximal end of system 100 to first and second ports 111A and 111B that are positioned adjacent to sensor 108. First and second biopsy needle guide lumens 110A and 110B may be accessible at proximal end of system 100 via any suitable device or configuration (e.g., one or more luers, a hub, access point within the bronchoscope, or the like). As shown in FIG. 1B, first and second biopsy needles 112A and 112B can be deployed such that a biopsy sample is acquired from a region adjacent to the sensor 107.

In some examples, first and second biopsy needles 112A and 112B can be bendable and can comprise a flexible material such as a polymer. In some examples, it may be desirable for first and second biopsy needles 112A and 112B to comprise polyether ether ketone (PEEK) such that the material is high strength, biocompatible, and able to hold a sharpened edge. In other examples, it may be desirable for first and second biopsy needles 112A and 112B to comprise a metal (e.g., steel or nitinol) cutting tip and a polymer shaft. In still other examples, first and second biopsy needles 112A and 112B can comprise of titanium alloy (e.g., nitinol) such that the needles 112A and 112B are able to sustain greater bend angles without kinking when compared to, e.g., steel needles because of the relative yield strengths of these materials. In further examples, first and second biopsy needles 112A and 112B can comprise combinations of these materials and/or other materials that provide desired properties and behaviors. In some examples, the materials of biopsy needles 112A and 112B and/or electrodes 108 may be selected so that they can be visible on a monitoring device (e.g., CT scan or ultrasound device).

In some examples, the biopsy needles 112A and 112B materials or geometry may also be selected so that the needle induces an impedance change between electrodes 108 of sensor 107 as the needle is deployed. In this way, the biopsy needles 112A and 112B may provide verification that the needle has entered the tumor location by changing the relative impedance in the targeted area. Additionally, one or more of the electrodes may be placed at on the one or more of the biopsy needles 112A and 112B (e.g., at the tip) and the impedance may be measured between these electrodes and electrodes 108 of sensor 107 as the needle is deployed. This impedance measurement may be used to determine the biopsy needle location, to determine which tissues are adjacent to the needle tip, or both. The information may be used to provide verification that the needle has reached the desired target location or to provide feedback to the user of the location of the needle relative to the biopsy tool through visual display feedback, audio feedback, or other feedback mechanism.

FIG. 1C is an end view of system 100 of FIGS. 1A and 1B, from the distal end D. As shown in FIG. 1C, camera 104 forms some but not all of the distal end D. The rest of the distal end D, circumscribed by outer housing 102 and not covered by the face of camera 104, is a light transmission region 114. It is through this region 114 that light from LED 106 or another light source can pass, to illuminate the area in front of camera 104. In examples, optical light guides (not shown) can be used to guide the light around the periphery of camera 104.

The components described so far can be used to position system 100 adjacent to an object in a narrow passage of a pulmonary system. FIG. 1D depicts a method for using system 100, according to the disclosure. The method of FIG. 1D includes inserting a bronchoscope is inserted into a pulmonary system (150). The bronchoscope includes a tool port, and the system 100 can be deployed there from (152). System 100 can be deployed when, for example, the bronchoscope is too large to access a region where an object is located in a pulmonary system. In an alternative, system 100 can be deployed independently, without a bronchoscope. In such an example, the process begins with deployment (152) rather than bronchoscope insertion (150). Once deployed, system 100 can be routed to that object by an operator, using optical guidance (154) provided by the system 100 and its associated camera 104 and LED 106. While system 100 is driven, using optical guidance from camera 104, sensing (156) is employed via the sensor 107 either in the form or ultrasonic sensing using an ultrasonic sensor or in the form of impedance sensing using one or more electrodes 108. Once sensing 156 has identified a region that includes an abnormality in the surrounding tissue, system 100 can be used to biopsy the abnormality (158). System 100 can then be retracted into the bronchoscope (if used) and system 100 can be removed from the pulmonary system.

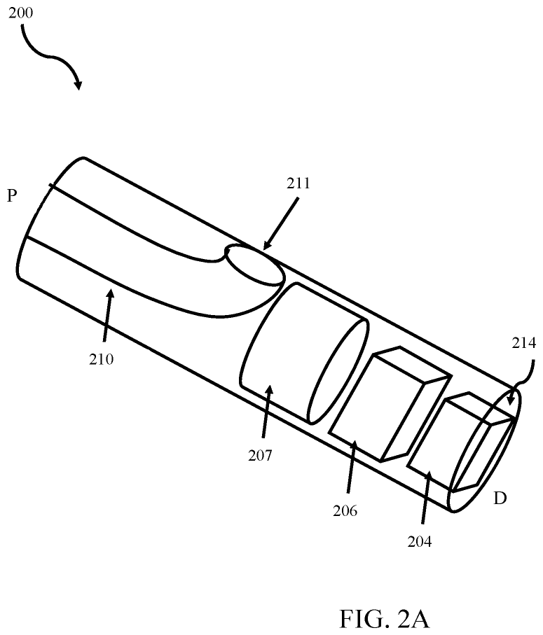

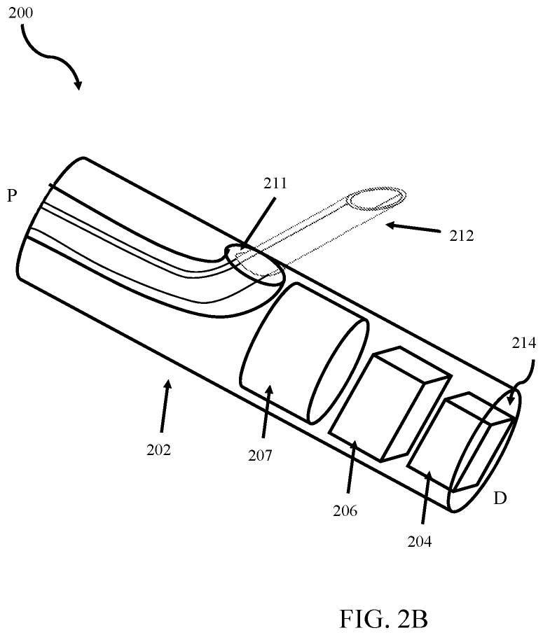

FIGS. 2A and 2B depict another example system, in which only a single biopsy needle is present. As shown in FIGS. 2A and 2B, similar components to those previously described with respect to FIGS. 1A-1C are given similar reference numerals, iterated by a factor of 100. This convention for identifying similar components is used throughout this application with respect to all of the figures.

Like the system 100 of FIGS. 1A-1C, system 200 of FIGS. 2A and 2B includes coaxially aligned structures including a camera 204, an LED 206, and sensor 207 (e.g., ultrasonic sensor or one or more electrodes), a biopsy needle guide lumen 210 terminating at an biopsy port 211, and a biopsy needle 212 that can be selectively deployed or retracted relative to biopsy needle guide lumen 210. Like the system 100 of FIGS. 1A-1C, system 200 of FIGS. 2A and 2B can be used to provide optical data and feedback so that an operator at the proximal end can guide it safely through relatively small pulmonary passages that could not be accessed by a bronchoscope. Finally, like the system 100 of FIGS. 1A-1C, system 200 can biopsy a region adjacent to sensor 207, which is configured to detect a change in acoustical or impedance properties indicative of a nodule, tumor, or other object being adjacent to sensor 207.

Because system 200 only includes one biopsy needle 212, an operator can manipulate system 200 from the proximal end to rotate it into a desired orientation before deploying biopsy needle 212. By making system 200 rotatable and including only a single biopsy needle 212, space requirements of system 200 are even less than the requirements of system 100 previously described with respect to FIGS. 1A and 1B. In examples, this permits for still further reduction in diameter of housing 202, as compared to system 100.

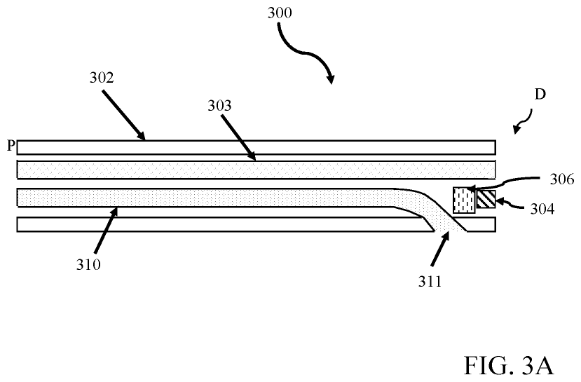

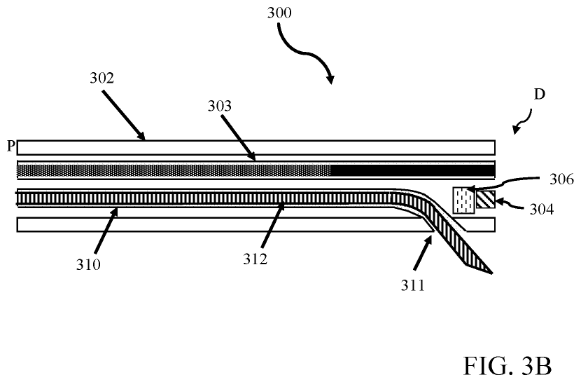

FIGS. 3A and 3B depict another example configured to accommodate a single biopsy needle and comprising a second lumen 303 to facilitate deployment of a sensor 307 (e.g., ultrasonic sensor or electrode sensor). Like system 100 of FIGS. 1A-1C, system 300 of FIGS. 3A and 3B includes a coaxially aligned camera 304, LED 306, and a biopsy needle guide lumen 310 terminating at an aperture 311, and a biopsy needle 312 that can be selectively deployed or retracted. Lumen 303 is arranged adjacent to camera 304 and LED 306 and extends within the region bounded by housing 302, from the proximal end P of housing 302 to the distal end D. A sensor 307 (e.g., ultrasonic sensor or electrode sensor) can be deployed via lumen 303. Sensor 307 can be any EBUS probe that fits within lumen 303 and is configured to detect a change in acoustical properties indicative of a nodule, tumor, or other object. Alternatively, sensor 307 can be an electrode probe configured to measure the impedance of tissue adjacent to distal end D.

Like the system 100 of FIGS. 1A-1C, system 300 of FIGS. 3A and 3B can be used to provide optical data and feedback so that an operator at the proximal end can guide system 300 safely through relatively small pulmonary passages that could not be accessed by a bronchoscope. Using the signal detected by sensor 307, a physician can determine that sensor 307 is adjacent to an object of interest for biopsy.

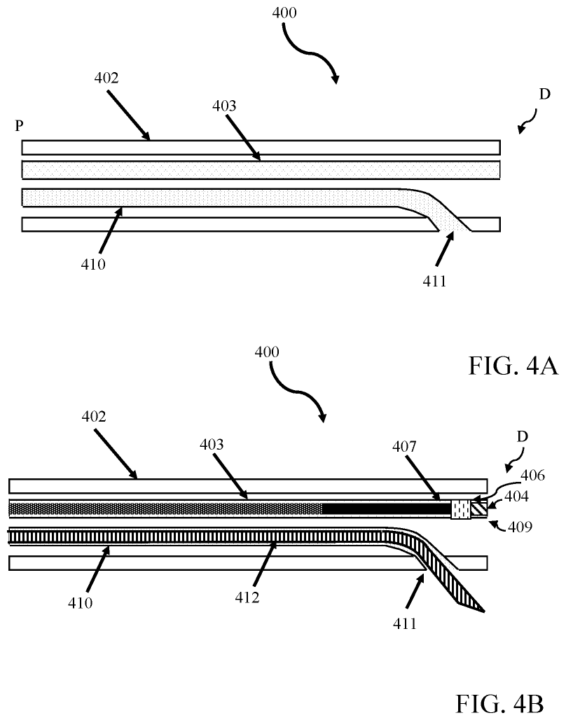

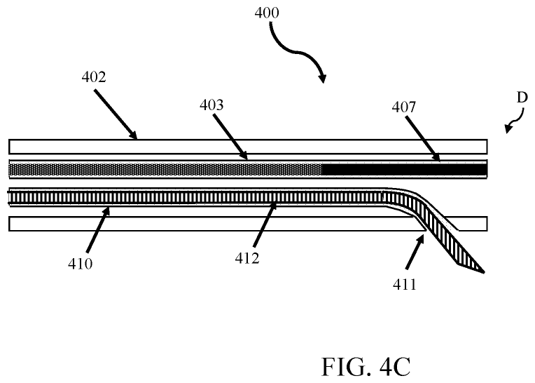

FIGS. 4A-4C depict another example, in which a camera and LED module 409 is interchangeable with a sensor. Like the system 100 of FIGS. 1A and 1B, system 400 of FIGS. 4A-4C includes a biopsy needle guide lumen 410 terminating at an aperture 411 in housing 402 before the distal end D of housing 402. A biopsy needle 412 can be selectively deployed or retracted via lumen 410. Lumen 403 is arranged adjacent to biopsy needle guide lumen 410 within housing 402, from the proximal end P of housing 402 to the distal end D. Like the system 300 of FIGS. 3A and 3B, a sensor 407 can be deployed via lumen 403 (see FIG. 4C). A module 409 containing a coaxially aligned camera 404 and LED 406 can be interchanged with sensor 407 for deployment via lumen 403 (see FIG. 4B).

Like system 100 of FIGS. 1A and 1B, system 400 of FIGS. 4A-4C can be used to provide optical data and feedback so that an operator at the proximal end can guide system 400 safely through relatively small pulmonary passages that could not be accessed by a bronchoscope. The operator can utilize module 409 to navigate to a region of interest. The operator can then interchange sensor 407 with module 409 in order to biopsy a region adjacent to sensor 407.

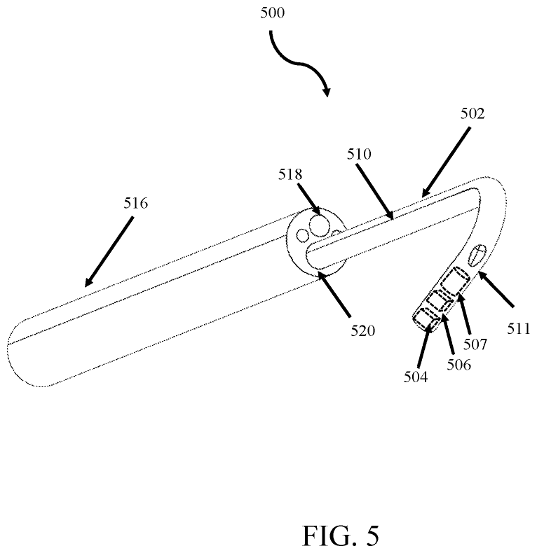

FIG. 5 is a perspective view of another example system 500, in which coaxially aligned camera 504, LED 506, and sensor 507 (e.g., ultrasonic sensor or electrode sensor) are arranged within a housing 502. System 500 extends from a bronchoscope 516. Bronchoscope 516 includes a distal end 518 having several ports. Tool port 520 is about 2 mm or less in diameter on standard bronchoscopes. When bronchoscope 516 reaches a portion of a pulmonary system where it can go no further due to its size, system 500 can be deployed to go further and potentially reach an object of interest for biopsy.

As shown in FIG. 5, system 500 can also selectively curve. Housing 502 can be made of a deformable material in examples, such as a polymer. Depending on the location of the curve and the extent of the curvature, there may be some misalignment of camera 504, LED 506, and sensor 507 from perfect coaxial alignment. Perfect alignment is not necessary, however, so long as light from LED 506 can pass around camera 504 either through housing 502 or another optically transmissive region (e.g., 114, 214). Curvature may be desirable where the passageway in the pulmonary system to the object of interest is curved. An operator can cause deformation of the housing 502 to follow the passageway without causing discomfort or injury to the patient by curving the housing 502 appropriately, in examples. The amount of curvature can be determined by the operator by using data from camera 504.

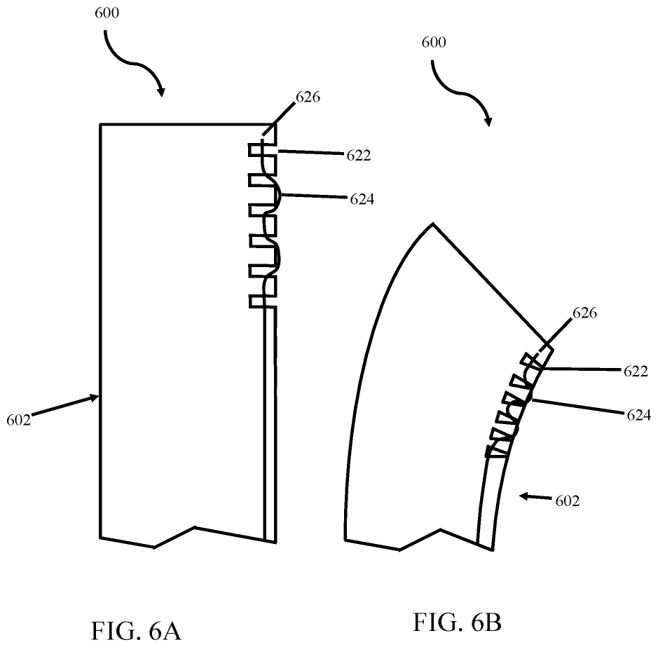

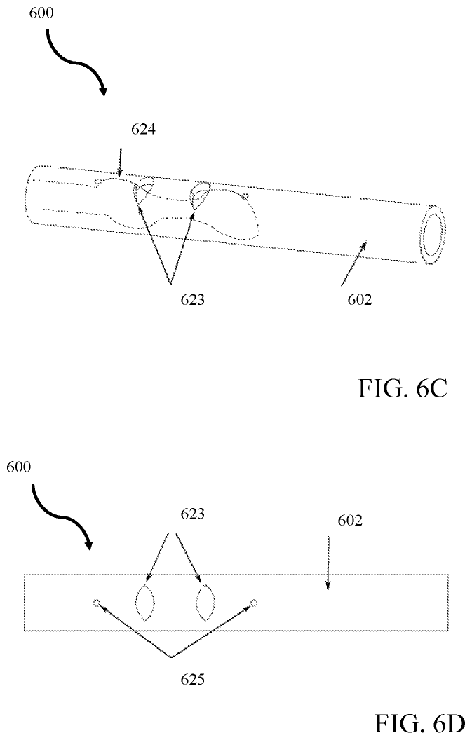

FIGS. 6A-6D depict an example mechanism for selectively causing curvature of a system as previously shown with respect to FIG. 5. An un-deflected view is shown in FIG. 6A. Housing 602 of a system 600 includes a plurality of apertures 622, threaded by cable 624. Cable 624 is anchored at anchor point 626. In order to cause curvature of housing 602, an operator can pull or otherwise manipulate the cable 624.

As shown in FIG. 6B, with the cable pulled, housing 602 bends, and apertures 622 are deformed to permit for more easy compression of the side of deflection. In examples, apertures 622 can be arranged on multiple sides of housing 602 (e.g., on two sides, spaced 180 degrees apart from each other around the outer circumference of housing 602), so that deflection in more than one direction is possible. Alternatively, in examples in which system 600 can be rotated, the operator can rotate housing 602 such that apertures 622 are arranged on the side to which the operator would like to turn. In such examples, a single set of apertures 622 arranged along one side of housing 602 can be sufficient to turn system 600 as needed.

FIGS. 6C and 6D depict an example geometry of apertures 622 for the threading of cable 624, in which the plurality of apertures 622 comprises at least two notches 623 and at least two holes 625. Notches 623 are arranged intermediate holes 625 and have a lens shape to facilitate bending of the lumen. Other shapes or configurations of notches 623 can be implemented in other examples. Holes 625 are small, in one example having a diameter that is slightly larger than cable 624. Holes 625 constrain the direction of bending and prevent twisting of housing 602 when it is being angulated. More or fewer apertures 622, of the configuration of notches 625 or holes 623, can be included in other examples, and the particular configuration, relative arrangement and spacings of apertures 622 can vary from the examples depicted according to a particular application or use of system 600. Additionally, apertures 622 of system 600 can be incorporated in other examples discussed herein.

In alternative examples, other systems could be used, rather than a cable 624 attached at an anchor point 626, to turn system 600. For example, housing 602 could include a memory-shape component such as a nitinol wire, or a piezoelectric actuator, or some other remotely operated system for turning the distal end of system 600. System 600 could also be steerable using either one or two angulation wires, similar to how traditional bronchoscopes are angulated, in alternative examples.

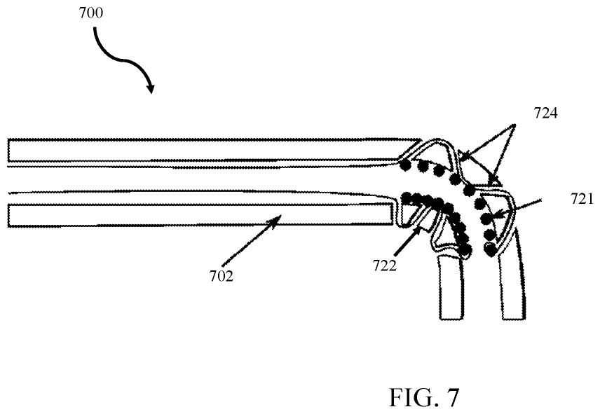

FIG. 7 depicts system 700, which additionally comprises at least one coil spring 721 to facilitate straightening and prevent kinking of housing 702 as it is selectively curved or otherwise directed. Coil spring 721 is arranged in the interior of housing 702 of system 700 and can have an outer diameter that is slightly smaller than an inner diameter of housing 702. Like housing 602 of system 600, housing 702 includes several notches 722 threaded by cable 724. Cable 724 is anchored at the distal end of coil spring system 721. Like in system 600, in order to cause curvature of housing 702, an operator can pull or otherwise manipulate cable 724. Coil spring system 721 prevents kinking of housing 702 while the housing is curved by providing internal support. Coil spring system 721 then facilitates straightening of housing 702 when the operator manipulates the cable to straighten system 700.

FIGS. 8A and 8B depict another example system 800, in which only a single biopsy needle is present. Like the system 100 of FIGS. 1A-1C, system 800 of FIGS. 8A and 8B includes coaxially aligned structures including a camera 804, an LED 806, and sensor 807, a biopsy needle guide lumen 810 (not shown) terminating at an biopsy port 811, and a biopsy needle 812 that can be selectively deployed or retracted relative to biopsy needle guide lumen 810. FIG. 8A illustrates a perspective view of system 800 and FIG. 8B illustrates a side view.

Like the system 100 of FIGS. 1A-1C, system 800 of FIGS. 2A and 2B can be used to provide optical data and feedback so that an operator at the proximal end can guide it safely through relatively small pulmonary passages that could not be accessed by a bronchoscope. Because system 800 only includes one biopsy needle 812 and biopsy needle guide lumen 810, an operator can manipulate system 800 from the proximal end to rotate it into a desired orientation before deploying biopsy needle 812. By making system 800 rotatable and including only a single biopsy needle 812, space requirements of system 800 are even less than the requirements of system 100 previously described with respect to FIGS. 1A and 1B. In examples, this permits for still further reduction in diameter of housing 802, as compared to housing 102 of system 100.

Finally, like the system 100 of FIGS. 1A-1C, system 800 includes sensor 807. Sensor 807 includes a plurality of electrodes 808 configured to detect a change in impedance properties indicative of a nodule, tumor, or other object being adjacent to sensor 807. Electrodes 808 include a plurality of primary electrodes 808A and a plurality of secondary electrodes 808B. Each respective primary electrode 808A is paired with a respective secondary electrode 808B at the same radial position relative to housing 802.

FIG. 8C shows another embodiment in which a device 800 includes a similar housing 802, with a biopsy port 811 and corresponding biopsy needle 812. In the embodiment shown in FIG. 8C, the optical components 804, 806 are similar to those described above with respect to other embodiments. The sensors 807 and 808, however, are different from those described previously, in that they are arranged both on the housing 802 as well as on the needle 812.

The arrangement of sensors 807 on the housing 802 while other sensor(s) 808 are arranged on the needle 812 provides substantial benefits for detection of some types of lung conditions. Specifically, arranging at least one sensor 808 on the needle 812 facilitates detection of properties of the surrounding tissue even before a biopsy could be performed. For example, when needle 812 is advanced into a tissue, the electromechanical characteristics such as firmness, electrical resistivity, pH, or others can vary with depth. While the optical components 804 and 806 are useful in advancing the system 800 to the appropriate location to obtain a sample, the additional placement of sensor 808, either alone or in combination with sensors 807, can provide a way to detect those properties as a function of depth within the sample. In this way, when a physician wished to obtain a sample from a region such as a tumor, and when the sensor 808 detects that properties indicative of that tumor are found adjacent the needle 812, the needle can be even more accurately targeted to obtain the exact tissue of interest for biopsy (and, as an added benefit, less tissue can be removed when the needle 812 is in fact in a region that seems healthy).

In embodiments, sensor 808 can be a pressure sensor or ultrasonic sensor capable of determining the mechanical properties of adjacent tissue. Alternatively or additionally, sensor 808 can be an electronic sensor capable of detecting electrical parameters of the adjacent tissue. For some systems 800, it may be helpful to include two sensors 808 (e.g., anode and cathode, or emitter and sensor ultrasonic systems) to make measurements on the adjacent tissue. It is also contemplated that there could be both mechanical and electrical sensors 808 arranged on the same needle 812, in alternative embodiments, or that sensors 808 could be implemented that are capable of both mechanical and electrical modes of operation.

In embodiments, sensors 808 can be operated in coordination with sensors 807. For example, sensors 807 could detect the electrical or mechanical characteristics of a first portion of the lung that data from sensor 808 can be compared to. Additionally or alternatively, sensors 807 can be a ground or complementary counterpart to sensor 808 (e.g., sensors 807 and 808 could be anode and cathode, respectively).

In embodiments, sensor 808 can be positioned proximate the tip of needle 812 such that the data acquired by sensor 808 is indicative of the electrical and/or mechanical properties of tissue very close to the tip. A sensor 808 is “proximate” to the tip of needle 812 if, in normal use, it would be expected that both the sensor 808 and the tip of needle 812 would be exposed by passing through the aperture 811 into the tissue.

In alternative examples, similar systems could be employed in other contexts. For example, rather than being used in a pulmonary system, a similar device could be sized to fit through other passages such as vasculature. In those examples, the relative size of the outer housing could be different in order to accommodate the expected size of the passages through which that system will pass, and the port, device, or catheter from which the system will be deployed. Likewise, the size and materials making up the camera, and the intensity or wavelength of the light source, can be varied to match the application.

Various examples of systems, devices, and methods have been described herein. These examples are given only by way of example and are not intended to limit the scope of the claimed inventions. It should be appreciated, moreover, that the various features of the examples that have been described may be combined in various ways to produce numerous additional examples. Moreover, while various materials, dimensions, shapes, configurations and locations, etc. have been described for use with disclosed examples, others besides those disclosed may be utilized without exceeding the scope of the claimed inventions.

By integrating camera and illumination in a single device having a small enough diameter to be deployed from the standard tool port of a bronchoscope, access to objects that would normally require expensive ENB is provided. In addition, the potential hazards associated with transthoracic needle puncture are reduced. This system is affordable enough to be used at medical centers that do not currently have ENB systems.

Persons of ordinary skill in the relevant arts will recognize that the subject matter hereof may comprise fewer features than illustrated in any individual example described above. The examples described herein are not meant to be an exhaustive presentation of the ways in which the various features of the subject matter hereof may be combined. Accordingly, the examples are not mutually exclusive combinations of features; rather, the various examples can comprise a combination of different individual features selected from different individual examples, as understood by persons of ordinary skill in the art. Moreover, elements described with respect to one example can be implemented in other examples even when not described in such examples unless otherwise noted.

Although a dependent claim may refer in the claims to a specific combination with one or more other claims, other examples can also include a combination of the dependent claim with the subject matter of each other dependent claim or a combination of one or more features with other dependent or independent claims. Such combinations are proposed herein unless it is stated that a specific combination is not intended.

Any incorporation by reference of documents above is limited such that no subject matter is incorporated that is contrary to the explicit disclosure herein. Any incorporation by reference of documents above is further limited such that no claims included in the documents are incorporated by reference herein. Any incorporation by reference of documents above is yet further limited such that any definitions provided in the documents are not incorporated by reference herein unless expressly included herein.

For purposes of interpreting the claims, it is expressly intended that the provisions of 35 U.S.C. § 112(f) are not to be invoked unless the specific terms “means for” or “step for” are recited in a claim.

1. A lung biopsy system comprising:
an elongated housing extending from a proximal end to a distal end;
at least one biopsy needle guide lumen extending within the housing, the guide lumen configured to enable deployment of a biopsy needle and extending from a proximal portion of the housing to a biopsy port in the housing, the biopsy port being positioned proximal to the distal end;
a camera configured to be arranged in the housing to obtain optical data relating to a position of the system;
a light source configured to be arranged in the housing adjacent to the camera to produce illumination for the camera; and
one or more sensors positioned at the housing to measure a property of a surrounding tissue.

2. The lung biopsy system of claim 1, wherein the sensor is arranged proximate the biopsy port.

3. The lung biopsy system of claim 1, wherein the sensor is configured to measure an impedance of a surrounding tissue adjacent the housing.

4. The lung biopsy tool of claim 1, further comprising:
a needle extending through the biopsy needle guide, and
a sensor arranged proximate a tip of the needle.

5. The lung biopsy system of claim 1, wherein the one or more sensors positioned at the housing comprise at least one primary electrode and at least one secondary electrode, the primary and secondary electrode being configured to transmit an electrical signal there between using the surrounding tissue to complete an electrical pathway.

6. The lung biopsy system of claim 4, wherein the one or more sensors positioned at the housing include at least one primary sensor and the sensor arranged proximate the tip of the needle is a secondary sensor, and wherein the primary and secondary sensors are configured to transmit an electrical signal there between using the surrounding tissue to complete an electrical pathway.

7. The lung biopsy system of claim 5, wherein the primary sensor and the secondary sensor form an electrode pair, wherein the electrode pair is positioned at a same radial position along the housing relative to a longitudinal axis defined by the housing.

8. The lung biopsy system of claim 7, wherein the biopsy port and the electrode pair are positioned relative to each other such that a biopsy needle exiting the biopsy port will obtain a tissue sample from a region adjacent to the electrode pair where the electrode measured an impedance of the surrounding tissue.

9. The lung biopsy system of claim 1, wherein at least one sensor of the one or more sensors positioned at the housing is arranged distal to the biopsy port.

10. The lung biopsy system of claim 5, wherein at least one electrode of the one or more electrodes is positioned proximal to the biopsy port.

11. The lung biopsy system of claim 1, further comprising one or more electrical conductors electrically coupled to the one or more sensors positioned at the housing.

12. The lung biopsy system of claim 11, wherein the one or more electrical conductors comprise a flexible circuit.

13. The lung biopsy system of claim 11, wherein the one or more electrical conductors comprise electrical wires carried by the housing.

14. The lung biopsy system of claim 1, wherein the housing further comprises a light guide configured to route light from the light source to the distal end.

15. The lung biopsy system of claim 1, wherein the camera and the light source are affixed at or adjacent to the distal end of the housing.

16. The lung biopsy system of claim 1, further comprising a lumen within the housing extending from the proximal portion of the housing to the distal end, wherein the camera and the light source are deployed to the distal end of the housing via the lumen.

17. The lung biopsy system of claim 1, wherein the housing further comprises a plurality of apertures formed in an external surface thereof, and the system further comprises a cable configured to be threaded through the plurality of apertures to selectively deform the housing.

18. The lung biopsy system of claim 17, wherein the plurality of apertures comprise at least two notches and at least two holes, and wherein the at least two notches are arranged between the at least two holes.

19. The lung biopsy system of claim 17, wherein the system further comprises at least one coil spring arranged in the housing proximate the plurality of apertures.

20. The lung biopsy system of claim 17, wherein the housing is configured to be deployed from a bronchoscope tool port.

21. The lung biopsy system of claim 17, further comprising a biopsy needle comprising a flexible polymer shaft and a metal tip, movably disposed within the guide lumen.

22. A method comprising:
navigating a lung biopsy system through a pulmonary system of a patient, the lung biopsy system comprising:
an elongated housing extending from a proximal end to a distal end;
at least one biopsy needle guide lumen extending within the housing, the guide lumen configured to enable deployment of a biopsy needle and extending from a proximal portion of the housing to a biopsy port in the housing, the biopsy port being positioned proximal to the distal end;
a camera configured to be arranged in the housing to obtain optical data relating to a position of the system;
a light source configured to be arranged in the housing adjacent to the camera to produce illumination for the camera; and
one or more electrodes carried by the housing and positioned adjacent to the biopsy port, the one or more electrodes configured to measure an impedance of a surrounding tissue of a patient adjacent to the one or more electrodes;
using the one or more electrodes, measuring the impedance within the surrounding tissue to detect relative changes in the impedance; and
deploying a biopsy needle through the biopsy needle guide lumen and out the biopsy port, wherein a biopsy needle sensor is arranged proximate a tip of the biopsy needle, and
obtaining a tissue sample from the surrounding tissue that is adjacent to the one or more electrodes via the biopsy needle, based upon a determination that the sensor proximate the tip of the biopsy needle corresponds to a substance of interest.

23. The method of claim 22, further comprising:
introducing a bronchoscope into the pulmonary system of the patient; and
introducing the lung biopsy system through the bronchoscope and deploying the lung biopsy system through a tool port defined by the bronchoscope.

24. The method of claim 22, further comprising:
transmitting an oscillating voltage across at least one pair of electrodes of the one or more electrodes;
using the oscillating voltage to measure a first relative impedance of a first tissue region adjacent to the one or more electrodes;
repositioning the lung biopsy system within the pulmonary system of the patient;
retransmitting an oscillating voltage across at least one pair of electrodes of the one or more electrodes;
using the retransmitted oscillating voltage to measure a second relative impedance of a second tissue region adjacent to the one or more electrodes;
comparing the first relative impedance to the second relative impedance to determine the presence of an abnormality in the second tissue region; and
deploying the biopsy needle into the second tissue region to obtain the tissue sample.
